(19) United States
(12) Patent Application Publication
(10) Pub. No.: US 2023/0253116 A1
(43) Pub. Date: Aug. 10, 2023

(54) ESTIMATING PATIENT RISK OF CYTOKINE STORM USING BIOMARKERS

(71) Applicant: Siemens Healthcare Diagnostics Inc., Tarrytown, NY (US)

(72) Inventors: Vivek Singh, Princeton, NJ (US); Matthias Siebert, Marloffstein (DE); Ali Kamen, Skillman, NJ (US); Puneet Sharma, Princeton Junction, NJ (US); Ankur Kapoor, Plainsboro, NJ (US); Dorin Comaniciu, Princeton, NJ (US)

(21) Appl. No.: 18/004,309

(22) PCT Filed: Aug. 4, 2021
(86) PCT No.: PCT/EP2021/071783

§ 371 Date: Jan. 5, 2023

Related U.S. Application Data
(60) Provisional application No. 63/065,585, filed on Aug. 14, 2020.
(60) Provisional application No. 63/065,663, filed on Aug. 14, 2020.
(60) Provisional application No. 63/191,440, filed on May 21, 2021.

(51) Int. Cl. G16H 50/30

(52) U.S. Cl. CPC G16H 50/30

(57) ABSTRACT

Systems and methods for determining an assessment of a patient for a medical condition are provided. Input medical data of a patient is received. A vector representing a state of the patient is generated based on the input medical data. An assessment of the patient for a medical condition is determined using a machine learning based network based on the vector. The assessment of the patient is output.

This application claims the benefit of U.S. Provisional Application No. 63/065,585, filed Aug. 14, 2020, U.S. Provisional Application No. 63/065,663, filed Aug. 14, 2020, and U.S. Provisional Application No. 63/191,440, filed May 21, 2021, the disclosures of which are herein incorporated by reference in their entirety.

### TECHNICAL FIELD

The present invention relates generally to estimating patient risk of cytokine storm, and in particular to estimating patient risk of cytokine storm using knowledge graphs and/or biomarkers of the patient.

### BACKGROUND

A cytokine storm (also referred to as hypercytokinemia) is a physiological reaction in which the immune system of a patient releases an uncontrolled and excessive amount of pro-inflammatory signaling molecules called cytokines. The sudden release of cytokines in large quantities can cause multisystem organ failure and death. Cytokine storms can be caused by a variety of conditions, including viral infections, sepsis, leukemia, lymphoma, and MAS (macrophage activation syndrome). Recent studies have shown a correlation between cytokine storms and severe manifestations of COVID-19 (coronavirus disease 2019) requiring intensive care and causing organ damage and failure.

Conventional approaches for estimating patient risk of cytokine storm are unable to incorporate prior knowledge and relationships between various patient variables. For example, where a patient has a preexisting condition such as asthma or diabetes and such a condition is known to have a significant impact on the patient risk of cytokine storms, conventional approaches are unable to incorporate such prior knowledge and relationships. As a result, conventional approaches are unable to estimate patient risk of cytokine storm with sufficient sensitivity and specificity.

### BRIEF SUMMARY OF THE INVENTION

In accordance with one or more embodiments, systems and methods for determining an assessment of a patient for a medical condition are provided. Input medical data of a patient is received. A vector representing a state of the patient is generated based on the input medical data. An assessment of the patient for a medical condition is determined using a machine learning based network based on the vector. The assessment of the patient is output. In one embodiment, the medical condition is a cytokine storm.

In one embodiment, the assessment of the patient comprises a risk or severity score for the medical condition. In another embodiment, the assessment of the patient comprises a patient outcome. The patient outcome may be one or more of a survival time or a discharge time.

In one embodiment, a knowledge graph is computed based on the input medical data. Another vector representing a state of the patient is generated based on the knowledge graph. The assessment of the patient for the medical condition is determined based on the other vector.

In one embodiment, the vector representing the state of the patient is generated using a machine learning based encoder. The machine learning based network is trained using training data imputed using the machine learning based encoder.

In one embodiment, the input medical data may comprise biomarkers of the patient.

These and other advantages of the invention will be apparent to those of ordinary skill in the art by reference to the following detailed description and the accompanying drawings.

### BRIEF DESCRIPTION OF THE DRAWINGS

FIG. 1 shows a method for determining an assessment of a patient for a medical condition, in accordance with one or more embodiments;

FIG. 2 shows a workflow for determining an assessment of a patient for a medical condition using machine learning based networks during an inference stage, in accordance with one or more embodiments;

FIG. 3 shows a workflow for training an encoder for each data type of the input medical data, in accordance with one or more embodiments;

FIG. 4 shows a user interface for determining an assessment of a patient, in accordance with one or more embodiments;

FIG. 5 shows a workflow for training machine learning based networks for determining an assessment of a patient, in accordance with one or more embodiments

FIG. 6 shows an exemplary artificial neural network that may be used to implement one or more embodiments;

FIG. 7 shows a convolutional neural network that may be used to implement one or more embodiments; and

FIG. 8 shows a high-level block diagram of a computer that may be used to implement one or more embodiments.

### DETAILED DESCRIPTION

The present invention generally relates to methods and systems for estimating patient risk of cytokine storm using knowledge graphs and/or biomarkers. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Embodiments described herein provide for the automatic assessment of a patient for a cytokine storm (or any other suitable medical condition). The assessment of the patient is determined based on a vector of latent features representing a state of the patient. The vector may be generated by encoding a knowledge graph of the patient and/or by encoding biomarker data of the patient. The knowledge graph incorporates prior knowledge and relations between input medical data of the patient, thereby enabling a more accurate and robust prediction for the assessment of the patient.

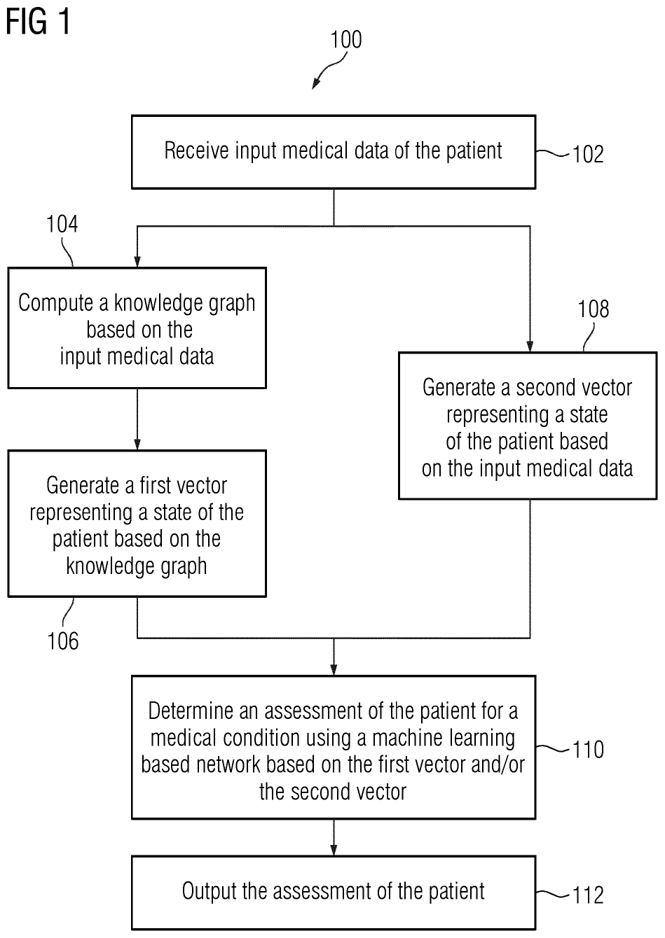

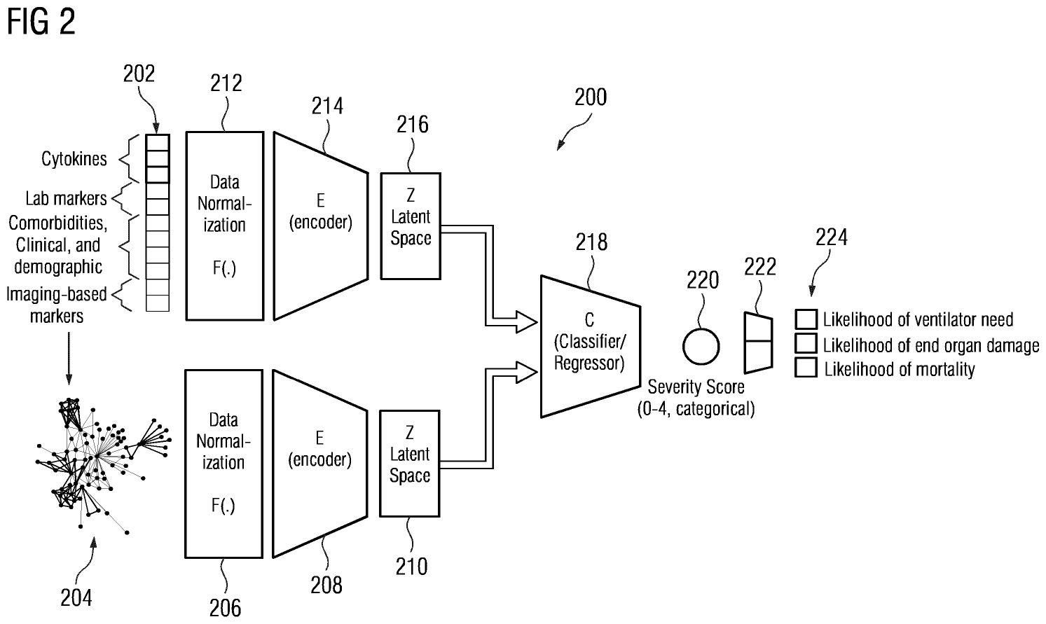

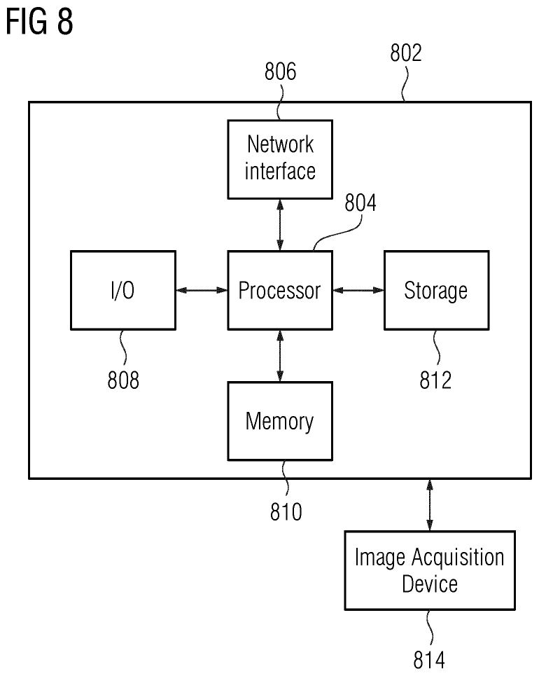

FIG. 1 shows a method 100 for determining an assessment of a patient for a medical condition, in accordance with one or more embodiments. Method 100 may be performed by one or more suitable computing devices, such as, e.g., computer 802 of FIG. 8. FIG. 2 shows a workflow 200 for determining an assessment of a patient for a medical condition using machine learning based networks during an inference stage, in accordance with one or more embodiments. FIG. 1 and FIG. 2 will be described together.

At step 102 of FIG. 1, input medical data of a patient is received. The input medical data may be any suitable medical data of the patient (including data derived therefrom). In one example, as shown in FIG. 2, the input medical data may be input medical data 202 comprising cytokines, lab markers, comorbidities, clinical, and demographic data, and imaging-based markers.

In one embodiment, the input medical data may comprise biomarkers of the patient. Biomarkers are objective, quantifiable characteristics of the patient. In one example, the biomarkers may include characteristics measured from the patient, such as, e.g., vital signs (e.g., temperatures, heart rate, respiration rate, blood pressure, blood oxygen saturation levels, etc.). In another example, the biomarkers may include laboratory results of an analysis of patient samples (e.g., blood), such as, e.g., cytokine levels (e.g., IL6 (interleukin 6) levels), IL8 (interleukin 8) levels, CRP (C-reactive protein) levels, neutrophil levels, eosinophil levels, lymphocyte levels, creatinine levels, D-dimer levels, LDH (lactate dehydrogenase) levels, INR (international normalized ratio), troponin-I levels, ferritin levels, BUN (blood urea nitrogen) levels, albumin levels, TNF-a (tumor necrosis factor alpha) levels, etc.). In a further example, the biomarkers may include characteristics extracted from medical imaging data of the patient, such as, e.g., ground glass volume percentage. The medical imaging data may comprise one or more 2D images or 3D volumes of any suitable modality, such as, e.g., computed tomography, magnetic resonance imaging, ultrasound, x-ray, etc. Such characteristics may be extracted from medical imaging data using any suitable approach.

In another embodiment, the input medical data may comprise patient history data of the patient. For example, the patient history data may include clinical data of the patient, comorbidities of the patient, family history of the patient, etc.

In another embodiment, the input medical data may comprise demographic information of the patient, such as, e.g., age, gender, ethnicity, blood type, location of birth or residence, etc.

The input medical data may be acquired in any suitable manner. For example, the input medical data may be acquired at time of admission of the patient at a hospital or may be acquired over a period of time while the patient is being monitored after being admitted to the hospital. The input medical data may be received by receiving the input medical data from a user (e.g., a clinician) as user input, by loading previously acquired medical data from a storage or memory of a computer system (e.g., an electronic health record system), or by receiving medical data that has been transmitted from a remote computer system.

Method 100 of FIG. 1 may then proceed to steps 104 and 106, to step 108, or to both. For instance, in one embodiment, method 100 may proceed to perform steps 104 and 106 without performing step 108. In another embodiment, method 100 may proceed to perform step 108 without performing steps 104 and 106. In another embodiment, method 100 may proceed to perform steps 104, 106, and 108. In this embodiment, steps 104 and 106 and step 108 may be performed in any order (i.e., steps 104 and 106 may be performed before, after, or simultaneously with step 108).

At step 104 of FIG. 1, a knowledge graph is computed based on the input medical data. The knowledge graph (also referred to as a semantic network) is a representation of the relationships between the input medical data. The knowledge graph is modelled as a directed graph where the input medical data is represented as nodes and the relationships between the input medical data is represented as edges connecting the nodes. The edges are associated with labels identifying the relationship between the nodes. In one example, as shown in FIG. 2, the knowledge graph may be knowledge graph 204 computed based on input medical data 202.

The knowledge graph may be computed according to any known approach. In one embodiment, the knowledge graph may be computed using graph convolution networks based on real or synthetically generated patient data. In one embodiment, the knowledge graph is computed using a meta-learning framework, where neural networks are used to optimize the graph embedding network to optimize the size and complexity of the embedding network, which ensures generalization on the validation sets.

The knowledge graph incorporates prior knowledge and relationships to define relationships between the input medical data. For example, the knowledge graph may relate protein concentrations to comorbidities, relate comorbidities to each other (as comorbidities differ between patients) and to the input medical data, include disease nodes in disease ontologies (e.g., human phenotype ontology) and define relationships between the disease nodes and patient comorbidities, and/or define relationships between patient demographics and habits (e.g., smoking) and comorbidities (e.g., chronic obstructive pulmonary disease).

In one embodiment, the knowledge graph captures relationships between confounding factors, such as, e.g., treatment and medication administered to the patient, to the input medical data. The knowledge graph relates the treatment and medication to proteins and comorbidities. The treatment and medication may be continually updated during the treatment to continually update the assessment of the patient determined according to method 100. The knowledge graph with continually updated treatment and medication may also be used for generating treatment plans. Exemplary relationships between treatment and medication and the input medical data include ventilation increases oxygen saturation levels, patients with asthma or autoimmune disease that are treated with immunosuppressants have lower cytokine levels, and relations relating to IL-6R (interleukin-6 receptor), such as, e.g., tocilizumab should not be combined with TNF-a inhibitors as it can lead to increased activity of CYP-450 (cytochrome P450) isoenzymes and thus cause corresponding pharmacological interactions, tocilizumab can lead to increased blood pressure as well as increased levels of liver enzymes (e.g., alanine aminotransferase), and sarilumab can lead to neutropenia and thrombozytopenia as well as increased levels of lipids and transaminases.

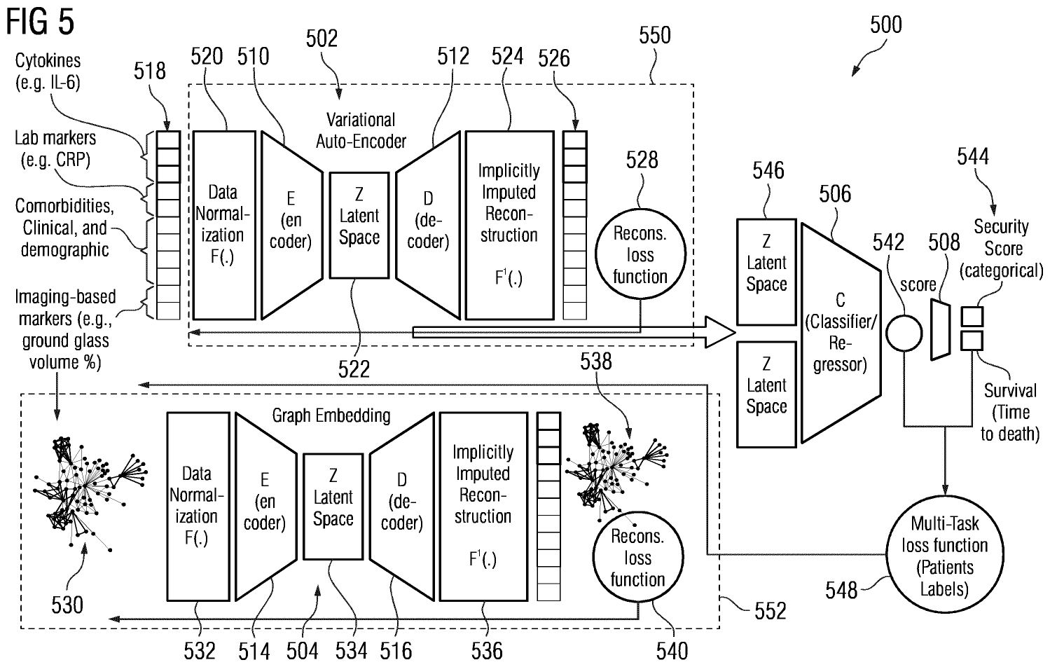

At step 106 of FIG. 1, a first vector representing a state of the patient is generated based on the knowledge graph. The first vector comprises latent features generated by encoding the knowledge graph using an encoder of a VAE (variational autoencoder) or any other suitable machine learning based network. In one embodiment, the knowledge graph is first normalized by applying a data normalization function and the encoder receives as input the normalized knowledge graph and generates as output the first vector of latent features in the z latent space. The data normalization function may be, for example, a z-normalization or standard zero-mean normalization. For example, as shown in FIG. 2, knowledge graph 204 is normalized by data normalization function 206 and the normalized knowledge graph is input into encoder E 208 to generate vector 210 of latent features in the z latent space. The encoder may be trained during a prior offline or training stage, as described below with respect to FIG. 5. Once trained, the encoder may be applied during an online or inference stage for generating the vector at step 106 of FIG. 1.

At step 108 of FIG. 1, a second vector representing a state of the patient is generated based on the input medical data. The second vector comprises latent features generated by encoding the input medical data using an encoder of a VAE or any other machine learning based network. In one embodiment, the input medical data is first normalized by applying a data normalization function and the encoder receives as input the normalized input medical data and generates as output the second vector of latent features in the z latent space. The data normalization function may be, for example, a z-normalization or standard zero-mean normalization. For example, as shown in FIG. 2, input medical data 202 is normalized by data normalization function 212 and the normalized input medical data is input into encoder E 214 to generate vector 216 of latent features in the z latent space. The encoder may be trained during a prior offline or training stage, as described below with respect to FIG. 5. Once trained, the encoder may be applied during an online or inference stage for generating the vector at step 108 of FIG. 1.

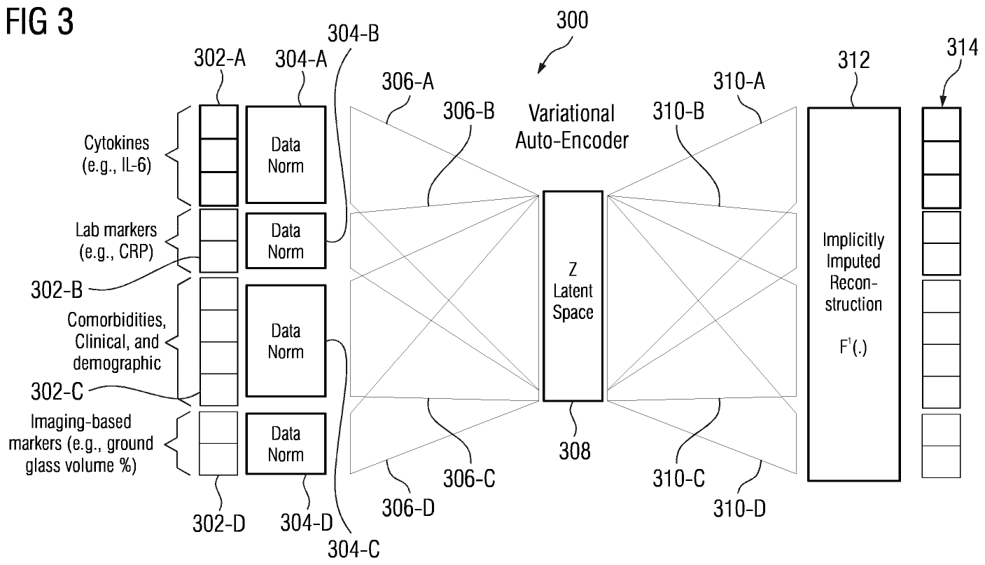

The input medical data may comprise a combination of heterogenous data types. For example, input medical data such as gender, age group, temperature, heart rate, and lab test results, may be categorical or discrete or may be continuous real-valued or only positive. To address this heterogeneity, in one embodiment, discrete values may be approximated as continuous values (prior to performing steps 104 and/or 108). In particular, the discrete values are represented using one hot vector representation and approximated as continuous values. The input medical data may then be combined in one large measurement vector. However, the approximation of discrete values as continuous values may be challenging, particularly in reconstruction tasks since the network trades off between predicting the continuous values accurately and estimating the approximated discrete variables sufficiently so that they go into the same category. In another embodiment, a different encoder may be applied for each data type of the input medical data. For example, FIG. 3 shows a workflow 300 for training an encoder for each data type of the input medical data, in accordance with one or more embodiments. Workflow 300 shows input medical data comprising cytokines 302-A, lab markers 302-B, comorbidities, clinical, and demographic 302-C, and imaging based markers 302-D of different types (collectively referred to as input medical data 302). Each input medical data 302 is normalized by a respective data normalization function 304-A, 304-B, 304-C, and 304-D and each normalized input medical data is encoded by a respective encoder 306-A, 306-B, 306-C, and 306-D to generate vector 308 of latent features in the z latent space. Vector 308 is decoded by respective decoders 310-A, 310-B, 310-C, and 310-D to reconstruct the normalized input medical data and then an implicitly imputed reconstruction function 312 is applied to the collective reconstructed normalized input medical data to reverse the normalization to generate reconstructed input medical data 314. Implicitly imputed reconstruction function 312 only penalizes the reconstruction of the data which was available in the original input medical data. The encoders and decoders in this embodiment may be trained using different reconstruction losses based on the data type. For example, a cross entropy loss may be used to train the encoder for categorical data, a mean squared or absolute error loss may be used to train the encoder for continuous values, etc.

At step 110 of FIG. 1, an assessment of the patient for a medical condition is determined using a machine learning based network based on the first vector and/or the second vector. In one embodiment, the medical condition is a cytokine storm. However, the medical condition may be any other suitable medical condition, such as, e.g., sepsis, ARDS (acute respiratory distress syndrome), multi-organ failure, etc.

The assessment of the patient may be any suitable assessment of the patient. In one embodiment, the assessment of the patient is a risk or severity score representing the risk or severity of the medical condition for the patient. The score may be a categorical score or a real valued score. For example, the categorical score may be low, medium, or high risk or severity. The real valued score may be on a scale from, e.g., 0 to 4, where 0 indicates no respiratory problems, 1 indicates mild or moderate respiratory problems, 2 indicates severe respiratory problems, 3 indicates severe respiratory problems with organ damage, and 4 indicates mortality. In another embodiment, the assessment of the patient is a patient outcome, such as, e.g., likelihood of ventilator need, the likelihood of end organ damage, the likelihood of mortality, the likelihood of a cytokine storm, the survival time, the discharge time, etc. The likelihood of the patient outcome may be for a period of time. For example, the likelihood of the patient outcome may be the likelihood of in-hospital mortality within 30 days.

The machine learning based network may be any suitable machine learning based network. In one embodiment, the machine learning based network is a classifier (or regressor) network. The classifier network may be implemented using a neural network. For example, the classifier may be implemented using a generative neural network with multiple output heads each corresponding to a different assessment. The machine learning based network may be trained during a prior offline or training stage, as described below with respect to FIG. 5. Once trained, the machine learning based network may be applied during an online or inference stage for determining the assessment of the patient at step 110 of FIG. 1.

The machine learning based network receives as input the first vector, the second vector, or a combination (e.g., concatenation, addition, or a combination thereof) of the first vector and the second vector and outputs the assessment of the patient. In one embodiment, the machine learning based network generates as output a score and the score is input into an additional network layer, which generates as output one or more patient outcomes. For example, as shown in FIG. 2, classifier/regressor C 218 receives as input a combination of vector 210 and vector 216 and generates as output severity score 220. Severity score 220 is input in additional network layer 222, which generates as output patient outcomes 224. In another embodiment, the machine learning based network generates as output one or more patient outcomes and the patient outcomes are input into an additional network layer, which generates as output a score. In another embodiment, the machine learning based network generates as output a score along with one or more patient outcomes (without using the additional network layer).

In step 112 of FIG. 1, the assessment of the patient is output. For example, the assessment of the patient can be output by displaying the assessment of the patient on a display device of a computer system, storing the assessment of the patient on a memory or storage of a computer system, or by transmitting the assessment of the patient to a remote computer system.

In one embodiment, method 100 may be repeated, e.g., for a particular number of iterations or for a particular amount of time (e.g., while the patient is admitted to the hospital) by continuously acquiring medical data of the patient to update the input medical data. In this embodiment, the assessment of the patient may be determined at step 110 based on the updated input medical data and any previously determined assessments of the patient.

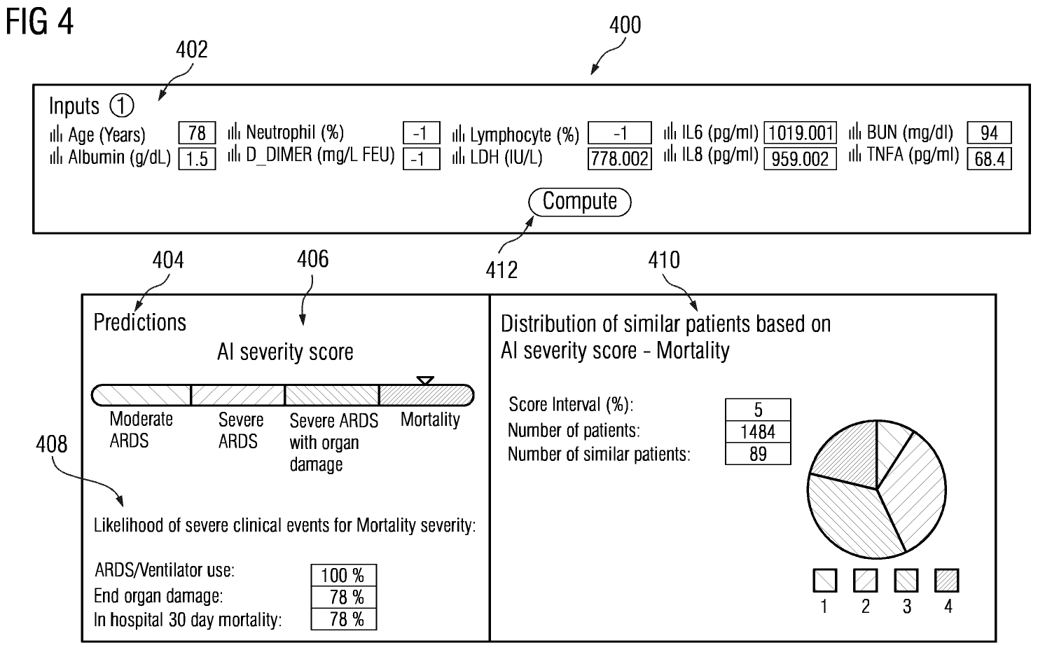

FIG. 4 shows a user interface 400 for determining an assessment of a patient, in accordance with one or more embodiments. A user (e.g., a clinician) interacts with user interface 400 to manually enter inputs 402 defining input medical data of a patient as user input and, in response to the user selecting the compute icon 412, predictions 404 providing an assessment of the patient for ARDS (acute respiratory distress syndrome) is output. Predictions 404 is determined according to method 100 of FIG. 1, where the input medical data received at step 102 of FIG. 1 is inputs 402 and the assessment of the patient output at step 112 of FIG. 1 is predictions 404. As shown in FIG. 4, predictions 404 comprises a severity score 406 and patient outcomes 408. Severity score 406 shows a categorical score indicating mortality and patient outcomes 408 show a likelihood of ARDS/ventilator use, end organ damage, and in-hospital 30 day mortality. User interface 400 also shows a distribution 410 of similar patients based on the severity score of mortality.

FIG. 5 shows a workflow 500 for training machine learning based networks for determining an assessment of a patient, in accordance with one or more embodiments. Workflow 500 shows VAE 502, VAE 504, and classifier 506. Workflow 500 is performed during a prior offline or training stage to train VAE 502, VAE 504, and classifier 506. Once trained, VAE 502, VAE 504, and classifier 506 are applied during an online or inference stage. For example, VAE 502 may be applied to perform step 106 of FIG. 1, VAE 504 may be applied at step 108 of FIG. 1, and classifier 506 may be applied at step 110 of FIG. 1.

VAE 502 comprises encoder E 510 and decoder D 512. VAE 502 is trained with training medical data 518 from a large amount of patient records (e.g., over 1,000 patient records). Training medical data 518 may be any suitable medical data. For example, training medical data 518 may be similar to the input medical data received at step 102 of FIG. 1. Training medical data 518 is normalized by data normalization function 520. Encoder E 510 is trained to encode the normalized training medical data to a vector 522 of latent features in the z latent space and decoder D 512 is trained to reconstruct the normalized training medical data from vector 522. Implicitly imputed reconstruction function 524 is applied to reverse the normalization of the reconstructed normalized training medical data to generate reconstructed training medical data 526. VAE 502 is trained according to reconstruction loss function 528.

VAE 504 comprises encoder E 514 and decoder D 516. VAE 504 is trained with knowledge graphs 530 generated from training medical data 518. Knowledge graph 530 is normalized by data normalization function 532. Encoder E 514 is trained to encode the normalized knowledge graph to a vector 534 of latent features in the z latent space and decoder D 516 is trained to reconstruct the normalized knowledge graph from vector 534. Implicitly imputed reconstruction function 536 is applied to reverse the normalization of the reconstructed normalized knowledge graph to generate reconstructed Knowle graph 538. VAE 504 is trained according to reconstruction loss function 540.

Classifier 506 is trained to receive vector 546 comprising vector 522, vector 534, or a combination or vector 522 and vector 534 and generate as output an assessment. In one embodiment, as shown in FIG. 5, assessment 542 may be a risk or severity score 542, which may be input into additional layer 508 to generate patient outcomes 544. In another embodiment, assessment 542 may be patient outcomes, which may be input into an additional layer to generate a risk or severity score. In another embodiment, assessment 542 is a risk or severity score and patient outcomes (without using the additional layer). Classifier 506 may be jointly trained with VAE 522 and/or VAE 504 using supervised learning according to multi-task loss function 548 using patient labels.

As shown in FIG. 5, workflow 500 comprises section 550 for training encoder E 510 to encode normalized training patient data to vector 522 and section 552 for training encoder E 514 for encoding normalized knowledge graph to vector 534. However, it should be understood that workflow 500 may be performed without section 550 or without section 552. For example, in one embodiment, where workflow 500 is performed to train machine learning networks to determine the assessment of the patient based on a first vector generated based on a knowledge graph without a second vector generated based on input medical data (e.g., by performing steps 104 and 106 of FIG. 1, but not step 108), workflow 500 is performed without section 550. In another embodiment, where workflow 500 is performed to train machine learning networks to determine the assessment of the patient based on a second vector generated based on input medical data without a first vector generated based on a knowledge graph (e.g., by performing step 108 of FIG. 1, but not steps 104 and 106), workflow 500 is performed without section 552. In another embodiment, where workflow 500 is performed to train machine learning networks to determine the assessment of the patient based on a first vector generated based on a knowledge graph and a second vector generated based on input medical data (e.g., by performing steps 104 and 106 and step 108 of FIG. 1), workflow 500 is performed with both section 550 and section 552.

In one embodiment, training medical data 518 may comprise partial data that does not include all medical data needed for training VAE 502, VAE 504, and/or classifier C 506. For example, lab tests are typically performed on an as needed basis and may not include all data needed for training. In one embodiment, missing training medical data may be imputed based on existing training medical data 518 (e.g., using standard imputation methods). In another embodiment, missing medical data may be imputed by VAE 502. In particular, encoder E 510 encodes the partial training medical data into vector 522 and decoder D 512 decodes vector 522 to reconstruct the training medical data to include imputed data. During training of VAE 502, weights associated with the missing medical data are not back propagated. In another embodiment, an additional mask vector may be input to classifier C 506 identifying true, collected medical data and imputed medical data (e.g., using standard methods). The classification network should recognize that imputed training medical data may not be accurate and is only an approximation. The identification of the imputed training medical data can be encoded within the loss function where, for example, the mean squared error is replaced by the log of the Mahalanobis distance where the variance is estimated from multiple reconstructions of the same sample.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, certain embodiments described herein are described with respect to methods and systems utilizing trained machine learning based networks (or models), as well as with respect to methods and systems for training machine learning based networks. Features, advantages or alternative embodiments herein can be assigned to the other claimed objects and vice versa. In other words, claims for methods and systems for training a machine learning based network can be improved with features described or claimed in context of the methods and systems for utilizing a trained machine learning based network, and vice versa.

In particular, the trained machine learning based networks applied in embodiments described herein can be adapted by the methods and systems for training the machine learning based networks. Furthermore, the input data of the trained machine learning based network can comprise advantageous features and embodiments of the training input data, and vice versa. Furthermore, the output data of the trained machine learning based network can comprise advantageous features and embodiments of the output training data, and vice versa.

In general, a trained machine learning based network mimics cognitive functions that humans associate with other human minds. In particular, by training based on training data, the trained machine learning based network is able to adapt to new circumstances and to detect and extrapolate patterns.

In general, parameters of a machine learning based network can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based network can be adapted iteratively by several steps of training.

In particular, a trained machine learning based network can comprise a neural network, a support vector machine, a decision tree, and/or a Bayesian network, and/or the trained machine learning based network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

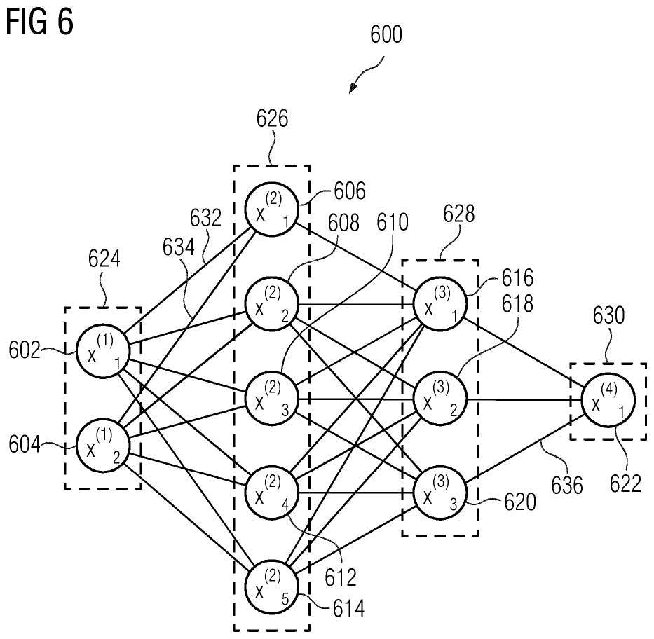

FIG. 6 shows an embodiment of an artificial neural network 600, in accordance with one or more embodiments. Alternative terms for “artificial neural network” are “neural network”, “artificial neural net” or “neural net”. Machine learning networks described herein, such as, e.g., the machine learning based networks utilized at steps 106, 108, and 110 of FIG. 1, encoder E 214, encoder E 208, and classifier C 218 of FIG. 2, encoders 306-A, 306-B, 306-C, and 306-D and decoders 310-A, 310-B, 310-C, and 310-D of FIG. 3, and VAE 502, VAE 504, and classifier C 506 of FIG. 5, may be implemented using artificial neural network 600.

The artificial neural network 600 comprises nodes 602-622 and edges 632, 634, . . . , 636, wherein each edge 632, 634, . . . , 636 is a directed connection from a first node 602-622 to a second node 602-622. In general, the first node 602-622 and the second node 602-622 are different nodes 602-622, it is also possible that the first node 602-622 and the second node 602-622 are identical. For example, in FIG. 6, the edge 632 is a directed connection from the node 602 to the node 606, and the edge 634 is a directed connection from the node 604 to the node 606. An edge 632, 634, . . . , 636 from a first node 602-622 to a second node 602-622 is also denoted as “ingoing edge” for the second node 602-622 and as “outgoing edge” for the first node 602-622.

In this embodiment, the nodes 602-622 of the artificial neural network 600 can be arranged in layers 624-630, wherein the layers can comprise an intrinsic order introduced by the edges 632, 634, . . . , 636 between the nodes 602-622. In particular, edges 632, 634, . . . , 636 can exist only between neighboring layers of nodes. In the embodiment shown in FIG. 6, there is an input layer 624 comprising only nodes 602 and 604 without an incoming edge, an output layer 630 comprising only node 622 without outgoing edges, and hidden layers 626, 628 in-between the input layer 624 and the output layer 630. In general, the number of hidden layers 626, 628 can be chosen arbitrarily. The number of nodes 602 and 604 within the input layer 624 usually relates to the number of input values of the neural network 600, and the number of nodes 622 within the output layer 630 usually relates to the number of output values of the neural network 600.

In particular, a (real) number can be assigned as a value to every node 602-622 of the neural network 600. Here, x⁽ⁿ⁾ᵢ denotes the value of the i-th node 602-622 of the n-th layer 624-630. The values of the nodes 602-622 of the input layer 624 are equivalent to the input values of the neural network 600, the value of the node 622 of the output layer 630 is equivalent to the output value of the neural network 600. Furthermore, each edge 632, 634, . . . , 636 can comprise a weight being a real number, in particular, the weight is a real number within the interval [−1, 1] or within the interval [0, 1]. Here, w^{(m,n)}_{i,j} denotes the weight of the edge between the i-th node 602-622 of the m-th layer 624-630 and the j-th node 602-622 of the n-th layer 624-630. Furthermore, the abbreviation w⁽ⁿ⁾_{i,j} is defined for the weight w^{(n,n+1)}_{i,j}.

In particular, to calculate the output values of the neural network 600, the input values are propagated through the neural network. In particular, the values of the nodes 602-622 of the (n+1)-th layer 624-630 can be calculated based on the values of the nodes 602-622 of the n-th layer 624-630 by

xⱼ⁽ⁿ⁺¹⁾=f(Σᵢxⱼ⁽ⁿ⁾·w_{i,j}⁽ⁿ⁾).

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 624 are given by the input of the neural network 600, wherein values of the first hidden layer 626 can be calculated based on the values of the input layer 624 of the neural network, wherein values of the second hidden layer 628 can be calculated based in the values of the first hidden layer 626, etc.

In order to set the values w^{(m,n)}_{i,j} for the edges, the neural network 600 has to be trained using training data. In particular, training data comprises training input data and training output data (denoted as tᵢ). For a training step, the neural network 600 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 600 (backpropagation algorithm). In particular, the weights are changed according to

w′_{i,j}⁽ⁿ⁾=w_{i,j}⁽ⁿ⁾−γ·δⱼ⁽ⁿ⁾·xᵢ⁽ⁿ⁾

wherein γ is a learning rate, and the numbers δ⁽ⁿ⁾ⱼ can be recursively calculated as

δⱼ⁽ⁿ⁾=(Σₖδₖ⁽ⁿ⁺¹⁾·w_{j,k}⁽ⁿ⁺¹⁾)·f′(Σᵢxᵢ⁽ⁿ⁾·w_{i,j}⁽ⁿ⁾)

based on δ⁽ⁿ⁺¹⁾ⱼ, if the (n+1)-th layer is not the output layer, and

δⱼ⁽ⁿ⁾=(xₖ⁽ⁿ⁺¹⁾−tⱼ⁽ⁿ⁺¹⁾)·f′(Σᵢxᵢ⁽ⁿ⁾·w_{i,j}⁽ⁿ⁾)

if the (n+1)-th layer is the output layer 630, wherein f′ is the first derivative of the activation function, and y⁽ⁿ⁺¹⁾ⱼ is the comparison training value for the j-th node of the output layer 630.

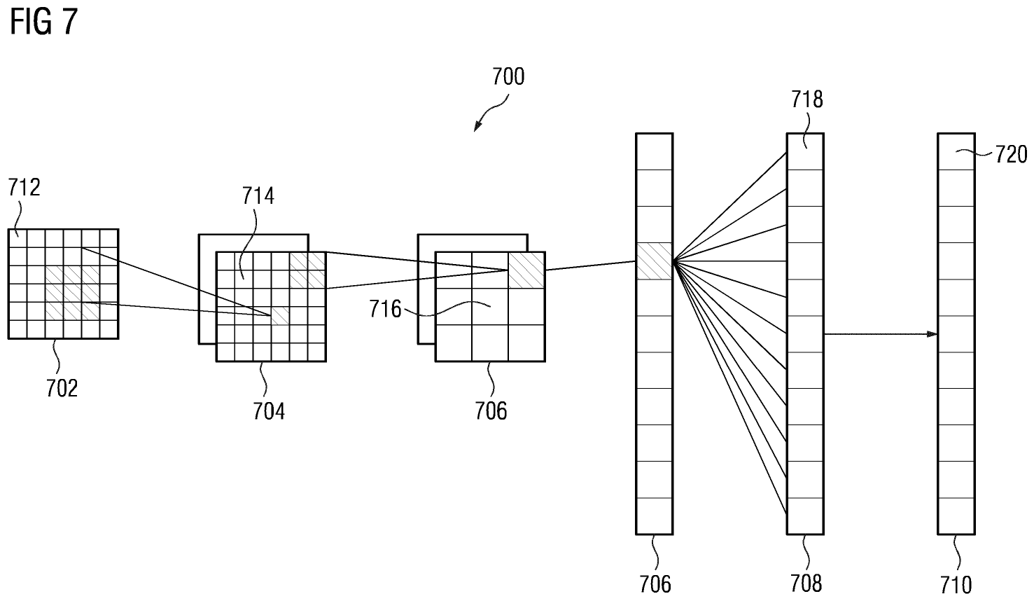

FIG. 7 shows a convolutional neural network 700, in accordance with one or more embodiments. Machine learning networks described herein, such as, e.g., the machine learning based networks utilized at steps 106, 108, and 110 of FIG. 1, encoder E 214, encoder E 208, and classifier C 218 of FIG. 2, encoders 306-A, 306-B, 306-C, and 306-D and decoders 310-A, 310-B, 310-C, and 310-D of FIG. 3, and VAE 502, VAE 504, and classifier C 506 of FIG. 5, may be implemented using convolutional neural network 700.

In the embodiment shown in FIG. 7, the convolutional neural network comprises 700 an input layer 702, a convolutional layer 704, a pooling layer 706, a fully connected layer 708, and an output layer 710. Alternatively, the convolutional neural network 700 can comprise several convolutional layers 704, several pooling layers 706, and several fully connected layers 708, as well as other types of layers. The order of the layers can be chosen arbitrarily, usually fully connected layers 708 are used as the last layers before the output layer 710.

In particular, within a convolutional neural network 700, the nodes 712-720 of one layer 702-710 can be considered to be arranged as a d-dimensional matrix or as a d-dimensional image. In particular, in the two-dimensional case the value of the node 712-720 indexed with i and j in the n-th layer 702-710 can be denoted as x⁽ⁿ_{)[i,j]}. However, the arrangement of the nodes 712-720 of one layer 702-710 does not have an effect on the calculations executed within the convolutional neural network 700 as such, since these are given solely by the structure and the weights of the edges.

In particular, a convolutional layer 704 is characterized by the structure and the weights of the incoming edges forming a convolution operation based on a certain number of kernels. In particular, the structure and the weights of the incoming edges are chosen such that the values x⁽ⁿ⁾ₖ of the nodes 714 of the convolutional layer 704 are calculated as a convolution x⁽ⁿ⁾ₖ=Kₖ*x⁽ⁿ⁻¹⁾ based on the values x⁽ⁿ⁻¹⁾ of the nodes 712 of the preceding layer 702, where the convolution * is defined in the two-dimensional case as

xₖ⁽ⁿ⁾[i,j]=(Kₖ*xⁿ⁻¹⁾[i,j]=Σ_{i′}Σ_{j′}Kₖ[i′,j′]·x⁽ⁿ⁻¹⁾[i−i′,j−j′].

Here the k-th kernel Kₖ is a d-dimensional matrix (in this embodiment a two-dimensional matrix), which is usually small compared to the number of nodes 712-718 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 712-720 in the respective layer 702-710. In particular, for a convolutional layer 704, the number of nodes 714 in the convolutional layer is equivalent to the number of nodes 712 in the preceding layer 702 multiplied with the number of kernels.

If the nodes 712 of the preceding layer 702 are arranged as a d-dimensional matrix, using a plurality of kernels can be interpreted as adding a further dimension (denoted as “depth” dimension), so that the nodes 714 of the convolutional layer 704 are arranged as a (d+1)-dimensional matrix. If the nodes 712 of the preceding layer 702 are already arranged as a (d+1)-dimensional matrix comprising a depth dimension, using a plurality of kernels can be interpreted as expanding along the depth dimension, so that the nodes 714 of the convolutional layer 704 are arranged also as a (d+1)-dimensional matrix, wherein the size of the (d+1)-dimensional matrix with respect to the depth dimension is by a factor of the number of kernels larger than in the preceding layer 702.

The advantage of using convolutional layers 704 is that spatially local correlation of the input data can exploited by enforcing a local connectivity pattern between nodes of adjacent layers, in particular by each node being connected to only a small region of the nodes of the preceding layer.

In embodiment shown in FIG. 7, the input layer 702 comprises 36 nodes 712, arranged as a two-dimensional 6×6 matrix. The convolutional layer 704 comprises 72 nodes 714, arranged as two two-dimensional 6×6 matrices, each of the two matrices being the result of a convolution of the values of the input layer with a kernel. Equivalently, the nodes 714 of the convolutional layer 704 can be interpreted as arranges as a three-dimensional 6×6×2 matrix, wherein the last dimension is the depth dimension.

A pooling layer 706 can be characterized by the structure and the weights of the incoming edges and the activation function of its nodes 716 forming a pooling operation based on a non-linear pooling function f. For example, in the two dimensional case the values x⁽ⁿ⁾ of the nodes 716 of the pooling layer 706 can be calculated based on the values x⁽ⁿ⁻¹⁾ of the nodes 714 of the preceding layer 704 as

x⁽ⁿ⁾[i,j]=f(x⁽ⁿ⁻¹⁾[id₁,jd₂z], . . . , x⁽ⁿ⁻¹⁾[id₁+d₁−1,jd₂+d₂−1])

In other words, by using a pooling layer 706, the number of nodes 714, 716 can be reduced, by replacing a number d₁·d₂ of neighboring nodes 714 in the preceding layer 704 with a single node 716 being calculated as a function of the values of said number of neighboring nodes in the pooling layer. In particular, the pooling function f can be the max-function, the average or the L2-Norm. In particular, for a pooling layer 706 the weights of the incoming edges are fixed and are not modified by training.

The advantage of using a pooling layer 706 is that the number of nodes 714, 716 and the number of parameters is reduced. This leads to the amount of computation in the network being reduced and to a control of overfitting.

In the embodiment shown in FIG. 7, the pooling layer 706 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 708 can be characterized by the fact that a majority, in particular, all edges between nodes 716 of the previous layer 706 and the nodes 718 of the fully-connected layer 708 are present, and wherein the weight of each of the edges can be adjusted individually.

In this embodiment, the nodes 716 of the preceding layer 706 of the fully-connected layer 708 are displayed both as two-dimensional matrices, and additionally as non-related nodes (indicated as a line of nodes, wherein the number of nodes was reduced for a better presentability). In this embodiment, the number of nodes 718 in the fully connected layer 708 is equal to the number of nodes 716 in the preceding layer 706. Alternatively, the number of nodes 716, 718 can differ.

Furthermore, in this embodiment, the values of the nodes 720 of the output layer 710 are determined by applying the Softmax function onto the values of the nodes 718 of the preceding layer 708. By applying the Softmax function, the sum the values of all nodes 720 of the output layer 710 is 1, and all values of all nodes 720 of the output layer are real numbers between 0 and 1.

A convolutional neural network 700 can also comprise a ReLU (rectified linear units) layer or activation layers with non-linear transfer functions including but not limited to leaky-RELU, sigmoid, tanh, parametric RELU, ELU and SELU. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 700 can be trained based on the backpropagation algorithm. For preventing overfitting, methods of regularization can be used, e.g. dropout of nodes 712-720, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1. Certain steps or functions of the methods and workflows described herein, including one or more of the steps or functions of FIG. 1, may be performed by a server or by another processor in a network-based cloud-computing system. Certain steps or functions of the methods and workflows described herein, including one or more of the steps of FIG. 1, may be performed by a client computer in a network-based cloud computing system. The steps or functions of the methods and workflows described herein, including one or more of the steps of FIG. 1, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 802 that may be used to implement systems, apparatus, and methods described herein is depicted in FIG. 8. Computer 802 includes a processor 804 operatively coupled to a data storage device 812 and a memory 810. Processor 804 controls the overall operation of computer 802 by executing computer program instructions that define such operations. The computer program instructions may be stored in data storage device 812, or other computer readable medium, and loaded into memory 810 when execution of the computer program instructions is desired. Thus, the method and workflow steps or functions of FIG. 1 can be defined by the computer program instructions stored in memory 810 and/or data storage device 812 and controlled by processor 804 executing the computer program instructions. For example, the computer program instructions can be implemented as computer executable code programmed by one skilled in the art to perform the method and workflow steps or functions of FIG. 1. Accordingly, by executing the computer program instructions, the processor 804 executes the method and workflow steps or functions of FIG. 1. Computer 802 may also include one or more network interfaces 806 for communicating with other devices via a network. Computer 802 may also include one or more input/output devices 808 that enable user interaction with computer 802 (e.g., display, keyboard, mouse, speakers, buttons, etc.).

Processor 804 may include both general and special purpose microprocessors, and may be the sole processor or one of multiple processors of computer 802. Processor 804 may include one or more central processing units (CPUs), for example. Processor 804, data storage device 812, and/or memory 810 may include, be supplemented by, or incorporated in, one or more application-specific integrated circuits (ASICs) and/or one or more field programmable gate arrays (FPGAs).

Data storage device 812 and memory 810 each include a tangible non-transitory computer readable storage medium. Data storage device 812, and memory 810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 808 may include peripherals, such as a printer, scanner, display screen, etc. For example, input/output devices 808 may include a display device such as a cathode ray tube (CRT) or liquid crystal display (LCD) monitor for displaying information to the user, a keyboard, and a pointing device such as a mouse or a trackball by which the user can provide input to computer 802.

An image acquisition device 814 can be connected to the computer 802 to input image data (e.g., medical images) to the computer 802. It is possible to implement the image acquisition device 814 and the computer 802 as one device. It is also possible that the image acquisition device 814 and the computer 802 communicate wirelessly through a network. In a possible embodiment, the computer 802 can be located remotely with respect to the image acquisition device 814.

Any or all of the systems and apparatus discussed herein may be implemented using one or more computers such as computer 802.

One skilled in the art will recognize that an implementation of an actual computer or computer system may have other structures and may contain other components as well, and that FIG. 8 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising:
receiving input medical data of a patient;
generating a vector representing a state of the patient based on the input medical data;
determining an assessment of the patient for a medical condition using a machine learning based network based on the vector; and
outputting the assessment of the patient.

2. The computer-implemented method of claim 1, wherein the medical condition is a cytokine storm.

3. The computer-implemented method of claim 1, wherein the assessment of the patient comprises a risk or severity score for the medical condition.

4. The computer-implemented method of claim 1, wherein the assessment of the patient comprises a patient outcome.

5. The computer-implemented method of claim 4, wherein the patient outcome comprises one or more of a survival time or a discharge time.

6. The computer-implemented method of claim 1, further comprising:
generating another vector representing a state of the patient based on a knowledge graph, the knowledge graph computed based on the input medical data,
wherein determining an assessment of the patient for a medical condition using a machine learning based network based on the vector comprises determining the assessment of the patient for the medical condition based on the other vector

7. The computer-implemented method of claim 1, wherein generating a vector representing a state of the patient based on the input medical data comprises:
generating the vector using a machine learning based encoder.

8. The computer-implemented method of claim 1, wherein the machine learning based network is trained using training data imputed using the machine learning based encoder.

9. The computer-implemented method of claim 1, wherein the input medical data comprises biomarkers of the patient.

10. An apparatus comprising:
means for receiving input medical data of a patient;
means for generating a vector representing a state of the patient based on the input medical data;
means for determining an assessment of the patient for a medical condition using a machine learning based network based on the vector; and
means for outputting the assessment of the patient.

11. The apparatus of claim 10, wherein the medical condition is a cytokine storm.

12. The apparatus of claim 10, wherein the assessment of the patient comprises a patient outcome.

13. The apparatus of claim 12, wherein the patient outcome comprises one or more of a survival time or a discharge time.

14. The apparatus of claim 10, further comprising:
generating another vector representing a state of the patient based on a knowledge graph, the knowledge graph computed based on the input medical data,
wherein the means for determining an assessment of the patient for a medical condition using a machine learning based network based on the vector comprises means for determining the assessment of the patient for the medical condition based on the other vector

15. A non-transitory computer readable medium storing computer program instructions, the computer program instructions when executed by a processor cause the processor to perform operations comprising:
receiving input medical data of a patient;
generating a vector representing a state of the patient based on the input medical data;
determining an assessment of the patient for a medical condition using a machine learning based network based on the vector; and
outputting the assessment of the patient.

16. The non-transitory computer readable medium of claim 15, wherein the assessment of the patient comprises a risk or severity score for the medical condition.

17. The non-transitory computer readable medium of claim 15, the operations further comprising:
generating another vector representing a state of the patient based on a knowledge graph, the knowledge graph computed based on the input medical data,
wherein determining an assessment of the patient for a medical condition using a machine learning based network based on the vector comprises determining the assessment of the patient for the medical condition based on the other vector

18. The non-transitory computer readable medium of claim 15, wherein generating a vector representing a state of the patient based on the input medical data comprises:
generating the vector using a machine learning based encoder.

19. The non-transitory computer readable medium of claim 15, wherein the machine learning based network is trained using training data imputed using the machine learning based encoder.

20. The non-transitory computer readable medium of claim 15, wherein the input medical data comprises biomarkers of the patient.
